(19) United States
(12) Patent Application Publication
(10) Pub. No.: US 2012/0171707 A1
(43) Pub. Date: Jul. 5, 2012

(54) ASSAY FOR INHIBITORS OF SUMO AND SUMO-TARGETED UBIQUITIN LIGASE PATHWAY

(75) Inventor: Gregory Prelich, New Rochelle, NY (US)

(73) Assignee: Albert Einstein College of Medicine of Yeshiva University, Bronx, NY (US)

(21) Appl. No.: 13/393,550

(22) PCT Filed: Aug. 13, 2010
(86) PCT No.: PCT/US10/02247

§ 371 Date: Mar. 20, 2012

Related U.S. Application Data
(60) Provisional application No. 61/276,393, filed on Sep. 11, 2009.

(51) Int. Cl. C12Q 1/02; C12N 1/19; C07K 14/00; C12Q 1/54

(52) U.S. Cl. 435/14; 435/29; 435/254.21; 530/350

(57) ABSTRACT

The present invention provides a method for determining a putative agent that inhibits the SUMO or the SUMO-targeted ubiquitin ligase pathway comprising the steps of contacting mutant MOT1 cells with the putative agent under conditions (1) permitting mutant MOT1 cell growth in the presence of a SUMO or SUMO-targeted ubiquitin ligase pathway inhibitor and (2) not permitting mutant MOT1 cell growth in the absence of a SUMO or SUMO-targeted ubiquitin ligase pathway inhibitor, and measuring cell growth.

### CROSS-REFERENCE TO RELATED APPLICATION

This application claims the benefit of U.S. Provisional Patent Application No. 61/276,393, filed Sep. 11, 2009, the content of which is hereby incorporated by reference into the subject application.

### STATEMENT OF GOVERNMENT SUPPORT

This invention was made with government support under grant number R01 GM52486 from the National Institutes of Health, U.S. Department of Health and Human Services. The government has certain rights in the invention.

### FIELD OF THE INVENTION

The present invention relates generally to chemical genetic strategies to screen for inhibitors of SUMOylation.

### BACKGROUND OF THE INVENTION

Throughout this application various publications are referred to in parenthesis. Full citations for these references may be found at the end of the specification. The disclosures of these publications are hereby incorporated by reference in their entirety into the subject application to more fully describe the art to which the subject invention pertains.

The ubiquitin-like family of proteins (Ubls) consists of ubiquitin itself and several structurally related proteins including SUMO (Small Ubiquitin-like MOdifier) (Kerscher et al. 2006). The Ubls are conjugated to target substrates via similar catalytic mechanisms as occurs in the ubiquitin pathway, but each Ubl is conjugated to substrates by its own dedicated pathway. Thus, the enzymes of the SUMO pathway are analogous to those that conjugate ubiquitin, but are encoded by different genes. In addition, although the Ubls are conjugated to substrates at lysine residues, similar to ubiquitin, the functions of Ubl conjugation are different from ubiquitylation, which often targets substrates for proteosome-mediated degradation.

SUMO is a small protein of 101 amino acids that is highly conserved in all eukaryotes and is required for viability of most eukaryotic cells (Kerscher et al. 2006). Similar to ubiquitin, SUMO is translated as an inactive precursor that is processed to the mature form by a SUMO-specific protease, which cleaves away the C-terminal four amino acids to expose the active C-terminal glycine. The mature SUMO is covalently attached to lysine residues of target proteins by a series of steps that requires E1, E2, and E3 enzymes that are analogous to those employed by the ubiquitin pathway (FIG. 1). The process is reversible, as SUMO can be removed from the substrate by the SUMO proteases. The enzymes of the SUMO pathway are specific for SUMO modification, as they do not catalyze processing, removal, or conjugation of ubiquitin or any other Ubls to target substrates.

The SUMO pathway is much simpler than that of ubiquitin (Table 1). In budding yeast, the core pathway consists of only ten proteins: SUMO, two E1 subunits (Aos1 and Uba2), a single E2 (Ubc9), four E3s (Siz1, Siz2, Mms21, and Zip3), and two SUMO proteases (Ulp1 and Ulp2). By comparison, the core ubiquitin machinery (Ub, E1s, E2s, E3s, and proteases) is encoded by at least 191 genes in yeast. Cellular localization of the Ulp1 SUMO protease requires the nuclear pore component Nup84, and SUMO-binding proteins are being identified, some of which likely function as downstream effectors of SUMO signaling. An example of a recently discovered downstream SUMO-binding effector, the Slx5-Slx8 heterodimer, is described below.

TABLE 1
Components of the SUMO pathway

| Species | Modifier | E1 | E2 | E3 | Protease |
|---|---|---|---|---|---|
| S. cerevisiae | SUMO | Uba2- | Ubc9 | Siz1, Siz2, | Ulp1, Ulp2 |
| | | Aos1 | | Mms21, | |
| | | | | Zip3 | |
| H. sapiens | SUMO- | SAE1- | UBC9 | PIAS family, | SENP1, |
| | 1, -2, -3, | SAE2 | | MMS21, | -2, -3, -5, -6, |
| | -4 | | | RanBP2, Pc2 | -7 |

The composition of the SUMO pathway in humans is very similar to that of yeast, with four SUMO genes, single E1 and E2s, seven reported E3s, and six protease genes. Conservation of the pathway extends beyond sequence similarity, down to the functional level, as human SUMO-1 can substitute for yeast SMT3 (SUMO). Thus, the mechanism of conjugation, the components, the sequences, and even the function of SUMO pathway genes are highly conserved from species as diverse as humans and yeast, providing strong incentive to take advantage of the power of yeast genetics to identify chemical inhibitors of the pathway.

Post-translational modification of proteins by ubiquitin and ubiquitin family members such as SUMO are highly conserved processes in eukaryotes that are essential for cell viability (Kerscher et al. 2006). The roles of ubiquitin in targeting proteins for proteosome-mediated degradation have been well-documented. Three outcomes for SUMOylated substrates have been documented. SUMO can: (1) directly alter protein activity, (2) alter cellular localization, and (3) affect protein stability (Johnson 2004), either by directly altering the structure of the target protein, or by providing a new surface for interaction with downstream effectors. Two facts attest to the broad biological significance of the SUMO pathway in vivo and the importance of developing effective drugs that target the pathway. First, proteomic approaches revealed that more than 500 of the ˜5,800 yeast proteins are SUMOylated, affecting fifteen major biological pathways (Makhnevych et al., 2009). This is undoubtedly a minimal estimate of the extent and broad importance of SUMOylation, as typically only a small proportion of the target protein is SUMOylated. Secondly, in humans, the targets of SUMOylation perform equally diverse functions, with SUMOylation being implicated as having important roles in cancer, viral infection, microbial pathogenicity, neural degeneration, and the inflammatory response, among others (Kerscher et al. 2006; Sarge and Park-Sarge, 2009).

A major obstacle in the field is that effective small molecule inhibitors of the SUMO pathway have not been identified to date. This is in stark contrast with its cousin ubiquitin, which is also highly conserved, essential for viability, targets many proteins involved in multiple biological processes, and with which it shares cross-regulatory mechanisms. Inhibitors of the ubiquitin-proteosome pathway (e.g. MG-132) have been invaluable tools for basic research to study the functions of ubiquitin in vivo, and the proteosome inhibitor Bortezomib/PS-341/Velcade is approved for treatment of patients with multiple myeloma and mantle cell lymphoma. By analogy with Bortezomib, inhibitors of the SUMO pathway have enormous potential impact for treatment of human genetic, metabolic, and infectious diseases. To date, there is no assay able to screen for SUMO inhibitors using chemical genetic strategies since screening for inhibitors of SUMOylation in vivo is difficult due to the lack of a strong non-lethal phenotype. The present invention addresses this need.

### SUMMARY OF THE INVENTION

The present invention provides a method for determining a putative agent that inhibits the SUMO or the SUMO-targeted ubiquitin ligase pathway comprising the steps of contacting mutant MOT1 cells with the putative agent under conditions (1) permitting mutant MOT1 cell growth in the presence of a SUMO or SUMO-targeted ubiquitin ligase pathway inhibitor and (2) not permitting mutant MOT1 cell growth in the absence of a SUMO or SUMO-targeted ubiquitin ligase pathway inhibitor, and measuring cell growth.

The present invention further provides a mutant MOT1 cell which has mutant PDR5.

The present invention also provides an assay for determining inhibitors of the SUMO or SUMO-targeted ubiqutin ligase pathway comprising mutant MOT1 cells in a medium.

Additionally, the present invention provides for the agent identified by the method for determining a putative agent that inhibits the SUMO or SUMO-targeted ubiquitin ligase pathway or identified by the assay for determining inhibitors of the SUMO or SUMO-targeted ubiquitin ligase pathway.

The present invention provides for the use of mutant MOT1 cells for the screening of putative agents that inhibit the SUMO or the SUMO-targeted ubiquitin ligase pathway.

### BRIEF DESCRIPTION OF THE DRAWINGS

FIG. 1. Steps in the SUMO pathway. SUMO is conjugated to target substrates by a biochemically analogous pathway to that used by ubiquitin, but utilizing SUMO-specific enzymes. The steps include maturation by a protease and conjugation via E1, E2, and E3 enzymes. The process is reversible, as SUMO can be removed from substrates by SUMO-specific de-conjugating enzymes.

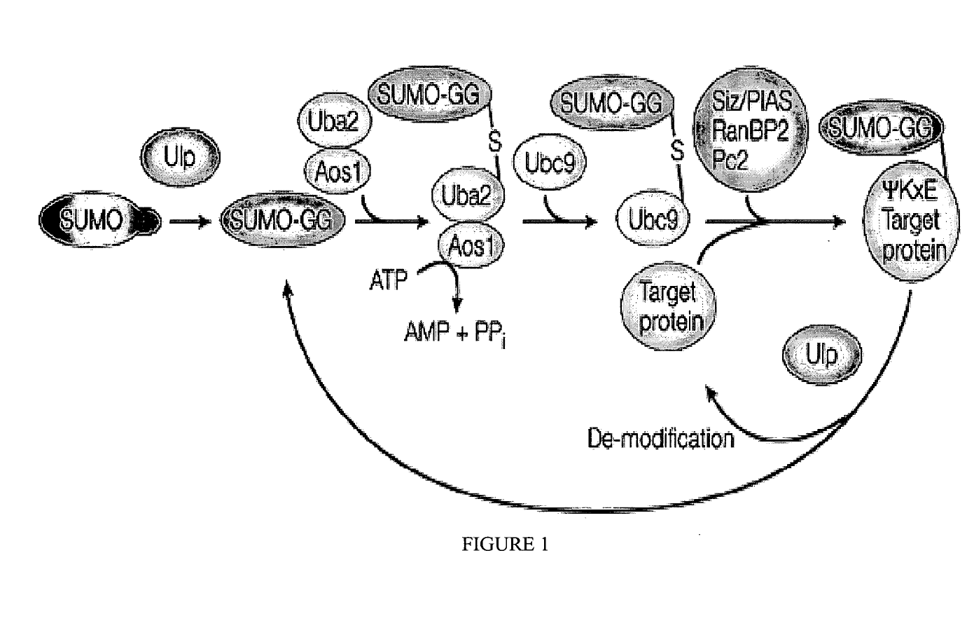

FIG. 2A-2B. Suppression of mot1-301 by SUMO and Ub pathway mutations. (A) The mot1-301 mutation causes four phenotypes that are easily scoreable in plate assays. All four phenotypes are strongly reversed by mutations in the SUMO-STUbL pathway. Suppression by siz1Δ siz2Δ (redundant SUMO E3s) is shown here. (B) The mot1-301 mutation was crossed into a panel of strains containing deletions of the non-essential ubiquitin E2s. Only ubc4Δ suppressed mot1-301, indicating that ubiquitin pathway also is needed to de-stabilize Mot1-301.

FIG. 3. The role of Slx5-Slx8 subunits. Slx5-Slx8 is a heterodimer, with each subunit containing RING domains. Slx5 contains a SUMO-Interacting Motif (SIM) that binds to SUMOylated substrates. Slx5 recruits Slx8, which has intrinsic ubiquitin E3 activity. In this way, Slx5-Slx8 preferentially targets SUMOylated substrates for ubiquitylation. Ubc4 is the ubiquitin E2 partner for Slx8.

FIG. 4. Growth curves. Growth of mot1-301 (OY566) and mot1-301 slx5Δ(OY570) strains shows the strong mot1-301 temperature-sensitive phenotype and suppression by slx5Δ. Strains were inoculated into 150 ml of YPD at the non-permissive temperature and growth measured by A600 in a Bioscreen C instrument. Duplicate assays are shown for both strains. The signal from ‘YPD only’ wells was not subtracted out.

FIG. 5. DMSO tolerance. Growth of the starting mot1-301 strain was tested in the presence of increasing concentration of DMSO at the permissive temperature. OY66 was inoculated into 150 ml of YPD and growth measured by A600 in a Bioscreen C instrument at the permissive temperature. Growth is barely affected by even 2% DMSO. The signal from ‘YPD only’ wells was not subtracted out from these readings.

### DETAILED DESCRIPTION OF THE INVENTION

The present invention provides a method for determining a putative agent that inhibits the SUMO or the SUMO-targeted ubiquitin ligase pathway comprising the steps of contacting mutant MOT1 cells with the putative agent under conditions (1) permitting mutant MOT1 cell growth in the presence of a SUMO or SUMO-targeted ubiquitin ligase pathway inhibitor and (2) not permitting mutant MOT1 cell growth in the absence of a SUMO or SUMO-targeted ubiquitin ligase pathway inhibitor, and measuring cell growth.

The SUMO and the SUMO-targeted ubiquitin ligase pathway are highly conserved among eukaryotic cells, including human and yeast, and are essential to the viability of most eukaryotic cells. The cells used in the present invention are preferably yeast cells, and most preferably are S. cerevisiae. MOT1 is a yeast gene which encodes an essential and conserved transcriptional regulator that uses ATP hydrolysis to remove TATA-binding protein from DNA. MOT1 is essential to cell viability and has functional homologues in eukaryotic cells, for example, human gene TAF-172.

The wild-type MOT1 sequence is: mtsrvsrldr qvilietgst qvvmmaadq mgdlakqhpe dilsllsrvy pfllvkkwet rvtaaravgg ivahapswdp nesdlvggtn egspldnaqv klehemkikl eeatqnnqln llqedhhlss lsdwklneil ksgkvllass mndynvlgka ddnirkqakt ddikqetsml nasdkanenk snankksarm lamarrkkkm sakntpkhpv ditessyskt llngknmtns aaslatspts nqlnpkleit eqadesklmi estvrplleq heivaglvwq fqgiyellld nlmsenweir hgaalglrel vkkhaygvsr vkgntreenn lrnsrsledl asrlltvfal drfgdyvydt vvapvresva qtlaallihl dstlsikifn cleqlvlqdp lqtglpnkiw eathggllgi ryfvsiktnf lfahgllenv vrivlyglnq sdddvqsvaa siltpitsef vklnnstiei lvttiwslla rldddisssv gsimdllakl cdhqevldil knkalehpse wsfkslvpkl ypflrhsiss vrravinlli aflsikddst knwlngkvfr lvfqnilleq npellqlsfd vyvallehyk vkhtektldh vfskhlqpil hllntpvgek gknyamesqy ilkpsqhyql hpekkrsise tttdsdipip knnehinida pmiagditll gldvilntri mgakafaltl smfqdstlqs fftnvlvrcl elpfstprml agiivsqfcs swlqkhpege klpsfvseif spvmnkqlln rdefpvfrel vpslkalrtq cqsllatfvd vgmlpqyklp nvaivvqget eagphafgve taekvygeyy dkmfksmnns ykllakkple dskhrvlmai nsakesaklr tgsilanyas sillfdglpl klnpiirslm dsvkeernek lqtmagesvv hliqqllenn kvnvsgkivk nlcgflcvdt sevpdfsvna eykekiltli kesnsiaaqd dinlakmsee aqlkrkggli tlkilfevlg psilqklpql rsilfdslsd heneeaskvd neqgqkivds fgvlralfpf msdslrssev ftrfpvlltf lrsnlsvfry saartfadla kissvevmay tireilplmn sagslsdrqg steliyhlsl smetdvlpyv iflivpllgr msdsnedvrn latttfasii klvpleagia dpkglpeelv asrererdfi qqmmdpskak pfklpiaika tlrkyqqdgv nwlaflnkyh lhgilcddmg lgktlqtici iasdqylrke dyektrsves ralpsliicp psltghwene fdqyapflkv vvyaggptvr ltlrpqlsda diivtsydva rndlavinkt eynycvldeg hiiknsqskl akavkeitan hrliltgtpi qnnvlelwsl fdflmpgflg tekmfqerfa kpiaasmsk tsskeqeagv lalealhkqv lpfmlrrlke dvlsdlppki iqdyycelgd lqkqlymdft kkqknvvekd ienseiadgk qhifqalqym rklcnhpalv lspnhpqlaq vqdylkqtgl dlhdiinapk lsalrtllfe cgigeedidk kasqdqnfpi qnvisqhral ifcqlkdmld mvendlflkky mpsvtymrld gsidprdrqk vvrkfnedps idclllttkv gglgInitga dtvifvehdw npmndlqamd rahrigqkkv vnvyriitkg tleekimglq kfkmniastv vnqqnsglas mdthqlldlf dpdnvtsqdn eeknngdsqa akgmediane tgltgkakea lgelkelwdp sqyeeeynld tfiktlr (SEQ ID NO:1).

Mot1-301, which is a mutant MOT1, is created via the substitution of MOT1 residue #1226, glutamic acid, with lysine. This substitution can be done via any method known in the art. The substitution can be either heterozygous, where only one copy of the gene is mutated, or homozygous, where both copies of the gene are mutated. The mutation results in cells with four phenotypic differences from the wild type: temperature-sensitive growth, poor growth on galactose-containing medium, increased transcription from the UAS-less suc2Δuas (-1900/-390) reporter, and altered transcription from the his4-912δ allele. SUMO and SUMO-targeted ubiquitin ligase pathway destabilize mot1-301. This destabilization, and subsequent degradation, results in an inability of mot1-301 cells to grow at non-permissive temperatures, for example, 37° C. as well as an inability of mot1-301 cells to grow on galactose-rich medium. A cell with a homozygous mot1-301 masks the effects of loss-of-function mutations in the SUMO or SUMO-targeted ubiquitin ligase pathway which would, in haploid strains, allow for the suppression of the SUMO or SUMO-targeted ubiquitin ligase pathway even in the absence of an inhibiting agent.

Alternatively, mutant MOT1 can be created by any method known in the art which yields a mutant cell whose growth is sensitive to specific variable which can be manipulated by any method known in the art.

PDR5 is a well known gene encoding a major drug efflux pump in yeast. The sequence for PDR5 can be found, among other places, through the National Center for Biotechnology Information. PDR5 can be mutated by any method known in the art, such as by deleting the entire PDR5 gene, or deleting or substituting portions of the PDR5 gene. Additionally, the mutation can be either heterozygous or homozygous. Mutations which are of use in the present invention are those which result in the absence or inoperability of the drug efflux pump. Preferably, the mutant PDR5 is a homozygous deletion of PDR5.

The present invention also provides for one or more controls to the method for determining a putative agent that inhibits the SUMO and SUMO-targeted ubiquitin ligase pathway. The control methods used may be one, or preferably all, of the following: (A) medium without cells, which is not contacted with the putative agent, and which is kept under conditions (1) permitting mutant MOT1 cell growth in the presence of a SUMO or SUMO-targeted ubiquitin ligase pathway inhibitor and (2) not permitting mutant MOT1 cell growth in the absence of a SUMO or SUMO-targeted ubiquitin ligase pathway inhibitor, and measured for cell growth; (B) mutant MOT1 cells which are not contacted with the putative agent, and are kept under conditions (1) permitting mutant MOT1 cell growth in the presence of a SUMO or SUMO-targeted ubiquitin ligase pathway inhibitor and (2) not permitting mutant MOT1 cell growth in the absence of a SUMO or SUMO-targeted ubiquitin ligase pathway inhibitor, and measured for cell growth; and/or (C) contacting mutant MOT1 cells which are mutant slx5 or slx8 with the putative agent under conditions (1) permitting mutant MOT1 cell growth in the presence of a SUMO or SUMO-targeted ubiquitin ligase pathway inhibitor and (2) not permitting mutant MOT1 cell growth in the absence of a SUMO or SUMO-targeted ubiquitin ligase pathway inhibitor, and measured for cell growth.

Slx5 and slx8 are SUMO-targeted ubiquitin ligases which destabilize mot1-301. The sequences for both slx5 and slx8 can be found, among other places, through the National Center for Biotechnology Information. Mutant slx5 and/or slx8 can be prepared by any method known in the art, including deleting the entire slx5 or slx8 gene, or deleting or substituting portions of the slx5 or slx8 gene. Mutations which are of use in the present invention are those which result in slx5 or slx8 which can not destabilize mot1-301, resulting in mot1-301 cells which have the ability to grow at otherwise non-permissive temperatures and the ability to grow on galactose-rich medium. Preferably, the mutant slx5 or slx8 is a homozygous mutation for either slx5 or slx8.

The putative agent in the present invention can be any chemical or biological agent such as a chemical, small compound, polypeptide, protein, protein fragment, or aptamer. Preferably, the putative agent is membrane-permeable. An aptamer may be a single stranded oligonucleotide or oligonucleotide analog that binds to a particular target molecule, such as a protein. More preferably, an aptamer may be a protein aptamer which consists of a variable peptide loop attached at both ends to a protein scaffold that interferes with protein interactions.

The medium in the present invention can be any medium known in the art, more preferably a nutrient medium. In one embodiment, the most preferable medium is a yeast rich media (YPD). In another embodiment, the most preferable medium is a galactose-rich medium.

The amount of time at which the cells are held under conditions (1) permitting mutant MOT1 cell growth in the presence of a SUMO or SUMO-targeted ubiquitin ligase pathway inhibitor and (2) not permitting mutant MOT1 cell growth in the absence of a SUMO or SUMO-targeted ubiquitin ligase pathway inhibitor can be any period which would, under conditions of optimal cell growth, provide sufficient time for multiple cell division cycles to occur. This period is between 6 and 48 hours, preferably between 12 and 36 hours. The most preferred time period is about 24 hours.

The temperature which (1) permits mutant MOT1 cell growth in the presence of a SUMO or SUMO-targeted ubiquitin ligase pathway inhibitor and (2) does not permit mutant MOT1 cell growth in the absence of a SUMO or SUMO-targeted ubiquitin ligase pathway inhibitor can be, in the present invention, any temperature at which a non-mutant MOT1 cell would undergo normal cell growth and cell cycles while a mutant MOT1 cell will not undergo cell growth or division. This is known as the non-permissive temperature. When the cells used in the present invention are yeast, the preferred non-permissive temperature is 37° C.

Cell growth in the present invention means that the cells have undergone reproduction, resulting in a greater number of cells than were originally present. Cell growth can be measured by any way known in the art. Cell growth after incubation can be measured, for example, by light absorbance or fluorescence. Any method of measuring growth by fluorescence known in the art can be used, such as Laser Induced Flourescence. Preferably, growth is measured by light absorbance. When the cells used in the present invention are yeast, the preferred method of measuring cell growth is by measuring light absorbance at 600 nm.

Cell growth indicates that the putative agent is an inhibitor of the SUMO or SUMO-targeted ubiquitin ligase pathway. A lack of cell growth, or attenuated cell growth, indicates that the putative agent is not an inhibitor of the SUMO or SUMO-targeted ubiquitin ligase pathway. Preferably, if a putative agent is not an inhibitor of the SUMO or SUMO-targeted ubiquitin ligase pathway, there will be a lack of cell growth.

The most preferred method for determining a putative agent that inhibits the SUMO or the SUMO-targeted ubiquitin ligase pathway comprises the steps of contacting homozygous mot1-301 cells which have homozygous mutant PDR5 with the putative agent at 37° C. for about 24 hours, and measuring cell growth by measuring absorbance at 600 nm. The most preferred method would additionally include at least one of the following controls: (A) keeping medium at 37° C. for about 24 hours, and measuring cell growth by measuring absorbance at 600 nm; (B) keeping homozygous mot1-301 cells which have homozygous mutant PDR5 in the absence of the putative agent at 37° C. for 24 hours, and measuring cell growth by measuring absorbance at 600 nm; and/or (C) contacting cells which are homozygous for mot1-301, homozygous mutant slx5 or slx8, and homozygous mutant PDR5 with the putative agent at 37° C. for 24 hours, and measuring cell growth by measuring absorbance at 600 nm.

The invention provides an assay for determining inhibitors of the SUMO or SUMO-targeted ubiquitin ligase pathway comprising mutant MOT1 cells in a medium. Preferably, the mutant MOT1 cells are homozygous for mot1-301 and homozygous mutant PDR5.

The present invention also provides for one or more controls to the assay for determining a putative agent that inhibits the SUMO and SUMO-targeted ubiquitin ligase pathway. The control may be one, or both of: (A) medium without cells; and/or (B) mutant MOT1 cells which have mutant slx5 or slx8 in a medium.

The medium in the present invention can be any medium known in the art, more preferably a nutrient medium. In one embodiment, the most preferable medium is a yeast rich media (YPD). In another embodiment, the most preferable medium is a galactose-rich medium.

The present invention could be performed with high throughput arrays, such as a 384-well plate format. Most preferably, the invention provides for an assay which is a high throughput array comprising wells containing (A) cells which are homozygous for mot1-301 and homozygous mutant PDR5, (B) control wells containing medium, and (C) control wells containing cells which are homozygous for mot1-301, are homozygous mutant PDR5, and are homozygous mutant slx5 or slx8 in a medium.

The present invention also provides for the agent identified by the method for determining a putative agent that inhibits the SUMO or the SUMO-targeted ubiquitin ligase pathway comprising the steps of contacting mutant MOT1 cells with the putative agent under conditions (1) permitting mutant MOT1 cell growth in the presence of a SUMO or SUMO-targeted ubiquitin ligase pathway inhibitor and (2) not permitting mutant MOT1 cell growth in the absence of a SUMO or SUMO-targeted ubiquitin ligase pathway inhibitor, and measuring cell growth. As described above, the mutant MOT1 cells are most preferably homozygous for mot1-301 and homozygous for mutant PDR5. Most preferably, the conditions under which the mutant MOT1 cells are contacted with the putative agent are 37° C. for 24 hours. Most preferably, cell growth is measured by measuring absorbance at 600 nm. Since cell growth indicates that the putative agent is an inhibitor of the SUMO or SUMO-targeted ubiquitin ligase pathway, agents identified by this method would be good candidates for further investigation as an effective therapeutic drug. Lack of cell growth, or attenuated cell growth, indicates that the putative agent is not an inhibitor of the SUMO or SUMO-targeted ubiquitin ligase pathway and would therefore not merit additional investigation as a possible therapeutic inhibitor of the SUMO or SUMO-targeted ubiquitin ligase pathway.

The present invention further provides for the use of homozygous mot1-301 cells with homozygous deletions of PDR5 for the screening of putative agents that inhibit the SUMO or the SUMO-targeted ubiquitin ligase pathway.

### EXPERIMENTAL DETAILS

### 1. Experimental Details

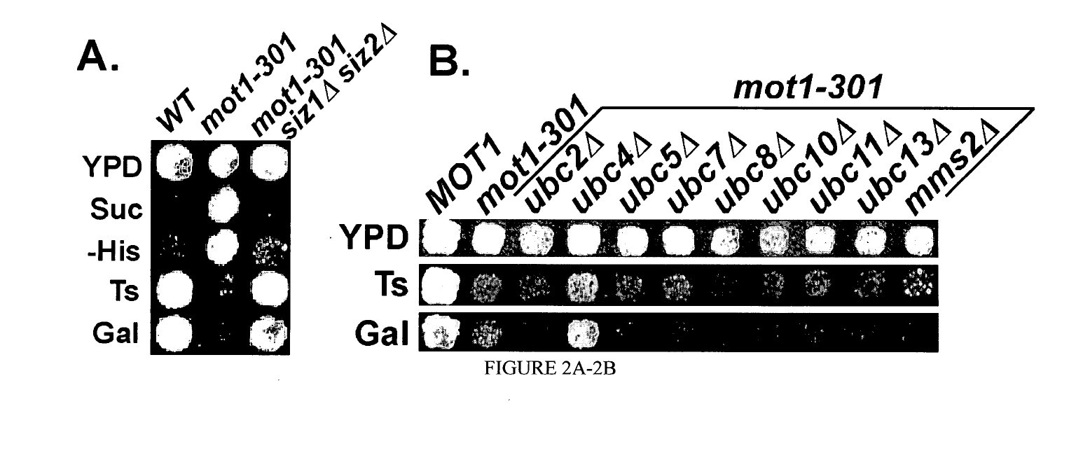

Primary assay development: mot1-301 strains display four mutant phenotypes (FIG. 2A), two of which (Ts- and Gal-) result in a loss of growth. The primary assay is to screen for small molecules that reverse the temperature-sensitive growth of a mot1-301 yeast strain. A major strength of this screen is that the desired compounds will cause an increase in growth rather than loss of growth, greatly reducing the false positives that typically arise from cell-based screens due to generalized toxicity. The mot1-301 starting strain is inoculated into 384-well plates in YPD medium, the compounds to be tested typically are added robotically by pinning in DMSO carrier, plates are incubated for 24 hours at the non-permissive temperature (37° C.), and growth measured by absorbance at 600 nm in a multi-well plate reader. Controls on each plate should include medium without any inoculated cells, untreated mot1-301 cells, and a mot1-301 slx5Δ strain.

In order to facilitate the chemical genetic screen, two challenges were overcome. One challenge was to block the isolation of spontaneous genomic suppressors of mot1-301. This was accomplished by constructing a diploid strain that is homozygous for mot1-301, effectively masking the effects of loss-of-function mutations in the SUMO pathway (or any other pathway) that would normally suppress in haploid strains. Thus the only spontaneous genomic suppressors obtained will be dominant gain-of-function mutations or intragenic revertants of mot1-301, which are extremely rare events. A mot1-301/mot1-301 diploid strain (OY566) has been constructed, and it has the same phenotypes as the haploid mot1-301 strain, except that the phenotypes are extremely stable. Secondly, for optimum effectiveness of the in vivo screen, the test inhibitors must enter the cells to be effective. Likelihood of success was increased by introducing homozygous deletions of PDR5, which is the major drug efflux pump in S. cerevisiae, into the OY566 starting strain.

### 2. Results

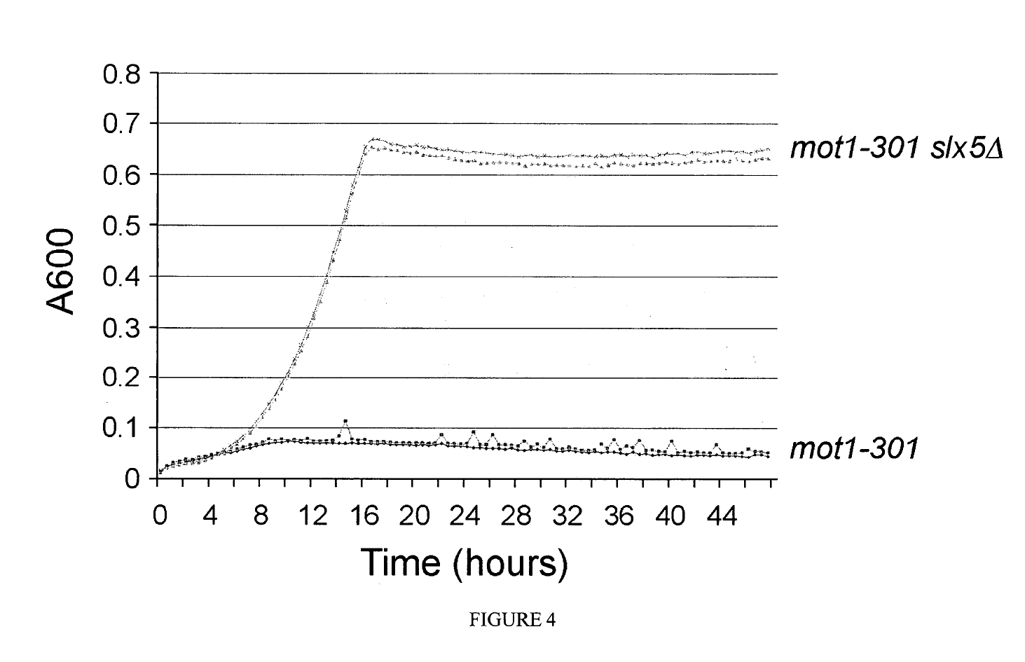

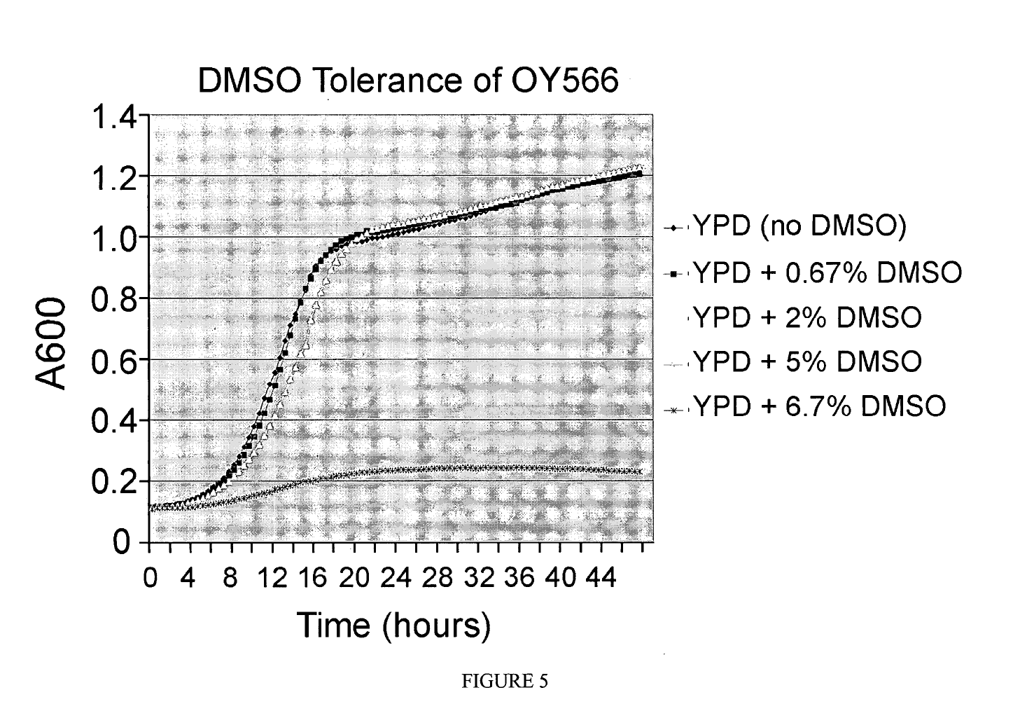

The necessary strains were created and preliminary growth assays were performed in 384-well format, comparing the mot1-301/mot1-301 strain (OY566) to a wild-type MOT1+/MOT1+ control (GY2171) and to a strain that contains a mot1-301 genomic suppressor in the SUMO pathway (mot1-301/mot1-301 slx5Δ/slx5Δ)(OY570). OY570 therefore provides a measure of the maximum anticipated suppression by blocking the STUbL pathway. Mimicking results previously observed in plate assays, in liquid growth curve assays the mot1-301 strain (OY566) displays a tight temperature-sensitive (Ts-) phenotype, which is strongly suppressed by slx5Δ (OY570)(FIG. 4). Growth of both a wild-type strain and the mot1-301 slx5Δ strain is unaffected by even 2% DMSO vehicle controls (FIG. 5), well within the final DMSO assay concentration of 0.2%. Reproducibility from well-to-well, plate-to-plate, and from day-to-day is strong, with a Z′ of 0.825, a coefficient of variability of 2.4%, and a signal-to-background ratio of 150 when the YPD medium background A600 reading is subtracted out. The assay is therefore robust and reproducible. The cost of the assay reagents is negligible, only requiring YPD medium, and stability of reagents is not an issue.

The mutations isolated cause no detectable phenotype in a MOT1+ background, yet they strongly suppress mot1-301, indicating that even minor perturbation of the pathway is detectable by this assay. Moreover, deletion of UBC4 (ubiquitin E2), SLX5, or SLX8 (ubiquitin E3), also suppresses mot1-301, indicating that defects in both the ubiquitin and the SUMO portions of the STUbL pathway can suppress mot1-301. Not only can mutations in the STUbL pathway suppress mot1-301, but suppressors are nearly limited to this pathway; >96% of the genomic suppressor mutations were in the STUbL pathway. This selection therefore is both sensitive and specific. Based on these results, it is anticipated that a chemical genetic screen for inhibitors of mot1-301, such as small compounds, will demonstrate the same specificity and sensitivity, targeting components of the STUbL pathway.

Hits that are detected in the primary assay can be subjected to a battery of standard tests to determine reproducibility and to determine whether those molecules directly target the SUMO or ubiquitin portions of the STUbL pathway.

### 3. Discussion

The SUMO pathway is highly conserved from yeast to humans and is essential for viability of most eukaryotic cells. Proteins are known to be SUMOylated, including proteins involved in such broad areas as cancer, microbial pathogenesis, neural degeneration, and the inflammatory response.

Despite the fact that the SUMO pathway targets many proteins, relatively few genetic selections have identified mutations in this pathway. A recent selection has identified every known member of the SUMO pathway, including some new genes that were not known to be part of the pathway. This selection is the most sensitive and best developed genetic system in any organism for studying the SUMO pathway.

Recent studies of the transcriptional regulator Mot1 in yeast revealed a SUMO pathway loss-of-function phenotype that provides the intellectual foundation for an assay to selectively identify small molecule inhibitors of the SUMO pathway. The mot1-301 mutation is strongly and specifically suppressed by loss-of-function genomic mutations in every step of the SUMO pathway. Inhibitors of the SUMO pathway, such as small molecules, are also expected to reverse the mot1-301 phenotypes. The primary assay is for inhibitors of the SUMO pathway which suppresses a mot1-301 mutation, causing growth at the non-permissive temperature. A robust and reliable assay that is insensitive to DMSO carrier has been developed in 384-well plate format. Two of the many advantages of this screen are that it screens for an increase in growth, not a decrease, thereby effectively eliminating common false positives that arise due to non-specific toxicity, and many components of the SUMO pathway are potential targets, increasing the likelihood of success. A panel of secondary assays can be used to identify those molecules or other inhibitors of SUMOylation, including testing the effect of the compounds on the SUMOylation status of Mot1 and other proteins in vivo, and testing the effect of the inhibitors in purified in vitro SUMOylation assays.

Chemical genetic strategies to screen for inhibitors of SUMOylation in vivo have been difficult due to the lack of a strong non-lethal phenotype. The present invention is based on a strong and unanticipated in vivo phenotype caused by loss-of-function mutations in the SUMO pathway. Mutations in every step of the SUMO pathway are found to suppress a temperature-sensitive mutation in the transcriptional regulator MOT1. Suppression is strong and specific, with >96% of the genomic suppressors occurring in SUMO pathway genes. This level of genetic saturation of the SUMO pathway is unprecedented, providing the best genetic system for studying SUMO in any organism. The present invention assay for small molecules that inhibit the pathway has the advantage that it can target any step in the pathway, including unsuspected steps, and that inactivating molecules can block the pathway by any mechanism, not simply by physical disruption of complexes. The present invention screening approach is therefore complementary to typical biochemical screening approaches, but is more comprehensive and with distinct advantages.

A selection for genomic suppressors of a mot1 mutation in S. cerevisiae (Wang et al. 2006) led to an unanticipated link with SUMO. The link between MOT1 and SUMO was not anticipated, as MOT1 encodes an essential and conserved transcriptional regulator that uses ATP hydrolysis to remove the TATA-binding protein from DNA, and no previous genetic or biochemical connections were established between MOT1 and SUMO. The specific mot1 mutation (mot1-301) causes four growth phenotypes, including temperature-sensitive growth on rich medium and the inability to grow on galactose-containing medium. Eighty-four genomic mutations that suppress mot1-301 were isolated, and >96% of those mutations (81/84) were in genes that encode components of the SUMO pathway (Table 2) (Wang et al. 2006). Mutations in the genes were identified that encode SUMO, the SUMO E1 and E2, the SUMO proteases, two other genes (SLX5 and SLX8) that were previously implicated as having a role in maintaining genome stability, and a gene that encodes a nuclear pore component (NUP84). Interestingly, SLX5, SLX8, and NUP84 were superficially linked to SUMOylation by previous studies; Slx5 was identified in a two-hybrid screen for SUMO-binding proteins, and Nup84 is required for nuclear localization of the Ulp1 SUMO protease. These previous clues, combined with finding these genes in a selection with the nearly the entire SUMO pathway, suggested that they might be legitimate components of the SUMO pathway. Extensive synthetic double mutant phenotypes were observed when slx5Δ and slx8Δ null alleles were crossed with mutations in known SUMO pathway genes, and slx5Δ and slx8Δ deletions resulted in an increase of SUMOylated proteins in a crude cell extract (Wang et al. 2006). In addition to these mutations that emerged from the genetic selection, subsequent directed tests revealed that deletion of the two redundant SUMO E3s SIZ1 and SIZ2 also suppressed mot1-301. Thus, mutations that affect every step of the SUMO pathway suppress mot1-301. This level of genetic saturation of the SUMO pathway is unprecedented.

TABLE 2
Genes containing mot1-301 suppressor mutations.

| # of | | |
|---|---|---|
| isolates | Gene | Function |
| 1 | SPT15 | TATA-Binding Protein |
| 2 | BUR6 | regulator of TBP |
| 4 | SMT3 | SUMO |
| 7 | UBA2 | SUMO E1 subunit |
| 5 | AOS1 | SUMO E1 subunit |
| 4 | UBC9 | SUMO E2 |
| 3 | ULP1 | SUMO protease |
| 3 | ULP2 | SUMO protease |
| 42 | SLX5 | STUbL subunit |
| 12 | SLX8 | STUbL subunit |
| 1 | NUP84 | nuclear pore subunit |

The mot1-301 suppressor mutations comprised 11 complementation groups. The suppressor genes fall into three groups: SPT15 and BUR6 (top), which were previously functionally linked to MOT1, known components of the SUMO pathway (middle), and SLX5, SLX8, and NUP84 (bottom), which were only superficially linked with the SUMO pathway previously.

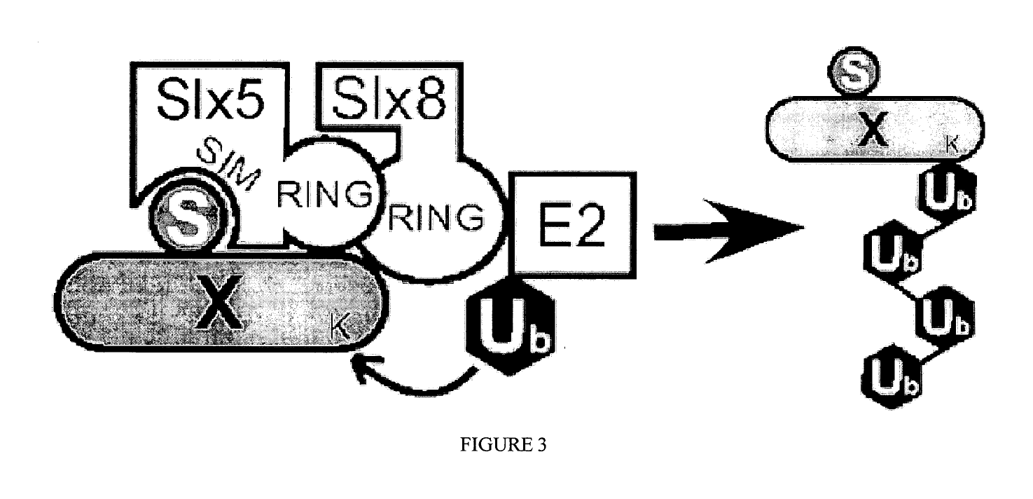

Studies from five labs (Mullen and Brill 2008; Prudden et al. 2007; Tatham et al. 2008; Uzunova et al. 2007; Xie et al. 2007) demonstrated that the Slx5-Slx8 heterodimer and its Sc. pombe and human orthologs have ubiquitin E3 ligase activity that preferentially targets SUMO conjugates for ubiquitylation; the Slx8 subunit of the Slx5-Slx8 heterodimer has ubiquitin E3 ligase activity, and it is preferentially recruited to SUMOylated substrates by an Slx5 SUMO-interacting motif (FIG. 3). In essence, the Slx5-Slx8 complex is a downstream effector that ubiquitinates SUMOylated substrates. Slx5-Slx8 and its orthologs have thus been termed STUbLs (SUMO-Targeted Ubiquitin Ligases). This finding forced a complete about-face in the field because SUMO had been suspected to compete with ubiquitin for some substrates, but the existence of STUbLs demonstrates that SUMOylation can also stimulate ubiquitylation of some proteins. Slx5-Slx8 cooperates with the Ubc4 ubiquitin E2 in vitro, and deletion of UBC4, but not genes encoding other ubiquitin E2s, also suppresses mot1-301 (FIG. 2B). The importance of these findings is accentuated by the fact that the Slx5-Slx5 human ortholog RNF4 is required for arsenic treatment in acute promyelocytic leukemia; RNF4 E3 ligase activity is required for SUMO-induced degradation of the PML-RARΔ fusion protein (Tatham et al. 2008).

To understand the role of STUbLs in vivo, it was important to identify additional physiologically relevant substrates, especially in yeast, where extensive genetic analysis is possible. Mot1-301 is a direct target of SUMOylation and that Mot1-301 is a very unstable protein due to its SUMO-, ubiquitin-, and proteosome-dependent degradation (Wang and Prelich 2009). Mutations in the SUMO pathway or in the sites of SUMOylation within Mot1 stabilize the protein, accounting for the suppression phenotype. Furthermore, Mot1-301 is targeted to a much greater extent than wild-type Mot1, but wild-type Mot1 becomes a stronger target under conditions that induce a mis-folded or defective state, hallmarks of a quality control function.

Selection for suppressors of mot1-301 is an extremely sensitive and specific method to detect defects in the SUMO/STUbL pathway, identifying every known step of the pathway. The mechanism of suppression is understood to a nearly unprecedented level in the SUMO field. The molecular defect is known (an unstable mutant protein) as are the target substrate (K101 and K109 of Mot1-301), the downstream effector (the Slx5-Slx8 ubiquitin E3), and its physiological role (protein quality control). This is a rare combination in the SUMO field which will allow an informed chemical genetic assay to identify small molecules that perturb the SUMO pathway.

The present assay has several advantages over a more traditional biochemical strategy for SUMO pathway inhibitors. Some of the advantages include: (1) the genetic rationale is strong, with the desired phenotype highly selective for disruption of the STUbL pathway; (2) hits in the screen are detected by an increase in growth, not a decrease, and therefore effectively eliminate common false positives due to non-specific toxicity; (3) slx5Δ serves as a strong positive control for the primary and secondary assays; (4) several secondary screens are available to confirm hits and determine whether they affect SUMOylation in vivo; (5) because many components of the SUMO and ubiquitin pathways are potential targets, the likelihood of success is high; (6) direct effects on the SUMO pathway can be assayed in vitro; and (7) the assay is inexpensive, requires no reagents beyond YPD medium, and is readily amenable to high-throughput implementation.

### REFERENCES

1. JOHNSON, E. S., 2004 Protein modification by SUMO. Ann. Rev. Biochem. 73: 355-382.
2. KERSCHER, O., R. FELBERBAUM and M. HOCHSTRASSER, 2006 Modification of proteins by ubiquitin and ubiquitin-like proteins. Annu Rev Cell Dev Biol 22: 159-180.
3. MAKHNEVYCH, T., SYDORSKYY, Y., XIN, X., SRIKUMAR, T., VIZEACOUMAR, F. J., et al. 2009 Global map of SUMO function revealed by protein-protein interaction and genetic networks. Mol. Cell 33: 124-135.
4. MULLEN, J. R., and S. J. BRILL, 2008 Activation of the Slx5-Slx8 ubiquitin ligase by poly-small ubiquitin-like modifier conjugates. J Biol Chem 283: 19912-19921.
5. PRUDDEN, J., S. PEBERNARD, G. RAFFA, D. A. SLAVIN, J. J. PERRY et al., 2007 SUMO-targeted ubiquitin ligases in genome stability. EMBO J 26: 4089-4101.
6. SARGE, D. D. and O. K. PARK-SARGE 2009 Sumoylation and human disease pathogenesis. Trends Biochem. Sci. 34: 200-205.
7. TATHAM, M. H., M. C. GEOFFROY, L. SHEN, A. PLECHANOVOVA, N. HATTERSLEY et al., 2008 RNF4 is a poly-SUMO-specific E3 ubiquitin ligase required for arsenic-induced PML degradation. Nat Cell Biol 10: 538-546.
8. UZUNOVA, K., K. GOTTSCHE, M. MITEVA, S. R. WEISSHAAR, C. GLANEMANN et al., 2007 Ubiquitin-dependent proteolytic control of SUMO conjugates. J Biol Chem 282: 34167-34175.
9. WANG, Z., G. M. JONES and G. PRELICH, 2006 Genetic analysis connects SLX5 and SLX8 to the SUMO pathway in Saccharomyces cerevisiae. Genetics 172: 1499-1509.
10. WANG, Z., and G. PRELICH, 2009 Quality control of a transcriptional regulator by SUMO-targeted degradation. Mol Cell Biol. 29: 1694-1706.
11. XIE, Y., O. KERSCHER, M. B. KROETZ, H. F. MCCONCHIE, P. SUNG et al., 2007 The yeast Hex3.Slx8 heterodimer is a ubiquitin ligase stimulated by substrate sumoylation. J Biol Chem 282: 34176-34184.
12. YAO, S., A. NEIMAN and G. PRELICH, 2000 BUR1 and BUR2 encode a divergent cyclin-dependent kinase-cyclin complex important for transcription in vivo. Mol Cell Biol 20: 7080-7087.

1. A method for determining a putative agent that inhibits the SUMO or the SUMO-targeted ubiquitin ligase pathway comprising the steps of contacting mutant MOT1 cells with the putative agent under conditions (1) permitting mutant MOT1 cell growth in the presence of a SUMO or SUMO-targeted ubiquitin ligase pathway inhibitor and (2) not permitting mutant MOT1 cell growth in the absence of a SUMO or SUMO-targeted ubiquitin ligase pathway inhibitor, and measuring cell growth.

2. The method of claim 1, wherein a condition (1) permitting mutant MOT1 cell growth in the presence of a SUMO or SUMO-targeted ubiquitin ligase pathway inhibitor and (2) not permitting mutant MOT1 cell growth in the absence of a SUMO or SUMO-targeted ubiquitin ligase pathway inhibitor comprises a non-permissive temperature.

3. The method of claim 1, wherein the non-permissive temperature is 37° C.

4. The method of claim 1, wherein a condition (1) permitting mutant MOT1 cell growth in the presence of a SUMO or SUMO-targeted ubiquitin ligase pathway inhibitor and (2) not permitting mutant MOT1 cell growth in the absence of a SUMO or SUMO-targeted ubiquitin ligase pathway inhibitor comprises a galactose-containing medium.

5. The method of claim 1, wherein the mutant MOT1 cells are homozygous for mot1-301.

6. The method of claim 1, wherein the mutant MOT1 cells further comprise mutant PDR5.

7. The method of claim 1, wherein the putative agent that inhibits the SUMO or the SUMO-targeted ubiquitin ligase pathway is a chemical, small molecule, polypeptide, protein, protein fragment, or aptamer.

8. The method of claim 1, additionally comprising a control wherein mutant MOT1 cells are kept under conditions (1) permitting mutant MOT1 cell growth in the presence of a SUMO or SUMO-targeted ubiquitin ligase pathway inhibitor and (2) not permitting mutant MOT1 cell growth in the absence of a SUMO or SUMO-targeted ubiquitin ligase pathway inhibitor, and measuring cell growth.

9. The method of claim 1, additionally comprising a control wherein mutant MOT1 cells which have mutant slx5 or slx8 are kept under conditions (1) permitting mutant MOT1 cell growth in the presence of a SUMO or SUMO-targeted ubiquitin ligase pathway inhibitor and (2) not permitting mutant MOT1 cell growth in the absence of a SUMO or SUMO-targeted ubiquitin ligase pathway inhibitor, and measuring growth.

10. The method of claim 1, wherein the mutant MOT1 cells are kept under conditions (1) permitting mutant MOT1 cell growth in the presence of a SUMO or SUMO-targeted ubiquitin ligase pathway inhibitor and (2) not permitting mutant MOT1 cell growth in the absence of a SUMO or SUMO-targeted ubiquitin ligase pathway inhibitor between 6 and 48 hours.

11. The method of claim 1, wherein cell growth is measured by measuring absorbance at 600 nm.

12. The method of claim 1, wherein cell growth indicates that the agent is an inhibitor of the SUMO or SUMO-targeted ubiquitin ligase pathway.

13. The method of claim 1, wherein a lack of cell growth indicates that the agent is not an inhibitor of the SUMO or SUMO-targeted ubiquitin ligase pathway.

14. A mutant MOT1 cell which has mutant PDR5.

15. An assay for determining inhibitors of the SUMO or SUMO-targeted ubiquitin ligase pathway comprising mutant MOT1 cells in a medium.

16. The assay of claim 15, wherein the mutant MOT1 cells are homozygous for mot1-301.

17. The assay of any of claim 15, wherein the mutant MOT1 cells further comprise mutant PDR5.

18. The assay of claim 15, wherein the assay additionally comprises at least one control.

19. The assay of claim 18, wherein the control is medium.

20. The assay of claim 18, wherein the control are mutant MOT1 cells which have mutant slx5 or slx8 in a medium.

21. The assay of claim 15, wherein the assay further comprises a putative agent that inhibits the SUMO or SUMO-targeted ubiquitin ligase pathway.

22. The agent identified by the method of claim 1.

23. (canceled)
